(19) United States
(12) Patent Application Publication
(10) Pub. No.: US 2008/0039814 A1
(43) Pub. Date: Feb. 14, 2008

(54) Disposable panty with built-in sanitary napkin or plug

(76) Inventor: Bert Jean, Brooklyn, NY (US)

Correspondence Address: SUNG I. OH, PROFESSIONAL LAW CORPORATION, 710 QUAIL VALLEY LANE, WEST COVINA, CA, 91791, US

(21) Appl. No.: 11/500,742

(22) Filed: Aug. 8, 2006

(51) Int. Cl. A61F 13/15

(52) U.S. Cl. 604/394; 604/402

(57) ABSTRACT

A disposable panty has a tampon-like element built thereinto. The panty also has two flaps that are releasably held together by hook-and-loop fastener material.

### TECHNICAL FIELD OF THE INVENTION

The present invention relates to the general art of wearing apparel, and to the particular field of undergarments.

### BACKGROUND OF THE INVENTION

Regular undergarments in current use are made of cotton and/or synthetic materials. The cotton and synthetic panties typically do not offer barrier protection. Often the synthetic panties have a cotton lined crotch to absorb vaginal discharges or perspiration. The absorbent/barrier properties of regular undergarments are minimal such that any vaginal discharge and/or heavy perspiration may strike through onto outer clothing (i.e., penetration of liquid from the interior to the exterior of the panty.)

Sanitary napkins are absorbent devices designed to receive and contain vaginal discharges such as menses. They are omnipresent items of modern society. In recent periods, disposable sanitary napkins designed to be held adjacent to the human body through the agency of a garment, such as an undergarment or panty, have effectively supplanted the more traditional type of sanitary napkin which required a specially designed belt for use.

Panty liners and feminine care sanitary napkins or pads used with regular undergarments have polyethylene backings that provide some barrier properties needed to prevent liquid strike through. However, if the vaginal discharge extends to the sides or the ends of the pads it can leak onto the undergarment. This leakage can stain the undergarment. Depending upon the amount of leakage, liquid may strike through or go around the undergarment to stain outer clothing and/or bedding. Women with heavy periods often use one or more maxi pads, double pads and/or tampons alone, or in combination, and change these pads and tampons frequently to prevent embarrassing, messy leakage and/or staining of outer clothing. In some cases, during their heaviest flow days, women will restrict their activities and stay home.

A majority of women experience some leakage of menses from their pads to their undergarments. This varies from being limited to a small number of pads leaking onto only the undergarment during light flow to leakage onto the wearer's outer clothing on almost half the pads worn during heavy flow. Normally this leakage occurs at the side of the product, although end leakage is also a problem. Placement of maxi pads and overnight pads in the crotch of regular undergarments shows that, at best, the pads lay on the leg elastic and, at worst, overhang the leg elastics. This causes side leakage onto the undergarment and possibly onto outer clothing. Typical leakage from the pads is caused by poor fit of the pad to the body, improper positioning of the pad by the user and lack of absorbency. Leakage from the undergarment onto the outer clothing is due to incompatibility between the pad width and panty crotch width and lack of barrier properties in the panty material around the edge portion of the pad.

Therefore, there is a need for an undergarment that can adequately protect a woman's clothing and still be convenient to wear during her menstrual cycle.

### SUMMARY OF THE INVENTION

The above-discussed disadvantages of the prior art are overcome by a disposable panty that has a tampon-like element built thereinto. The panty also has two flaps that are releasably held together by hook-and-loop fastener material so that the flaps can be quickly and easily opened so the garment can be easily removed without requiring it to be drawn over the wearer's legs. The flaps can also be moved with respect to each other whereby the size of the garment can be easily and accurately adjusted.

Using the garment embodying the present invention will permit a user to quickly and easily don the garment and accurately adjust the size thereof so it will remain in place to achieve its goal of a secure leak-proof fit.

With regard to disposable feature of the undergarment embodying the present invention, it is noted that the panties can be easily and quickly removed. This becomes especially important when the garment is soiled and the soilage could be spread and/or smeared if the garment had to be removed by sliding it down the length of one or both legs. The rapid opening feature of the garment embodying the present invention avoids this disadvantage

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWING FIGURES

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a perspective view of a disposable panty embodying the present invention in the open condition.

FIG. 2 is a side elevational view of the disposable panty of the present invention in the closed condition.

### DETAILED DESCRIPTION OF THE INVENTION

Referring to the figures, it can be understood that the present invention is embodied in a disposable lined panty 10 which is easily donned and removed and can be easily adjusted for a proper fit. Panty 10 comprises a body section 12 which has a first portion 14 that is a front portion when the body section is worn, a second portion 16 that is a rear portion when the body section is worn and a crotch portion 18 which connects the first portion to the second portion.

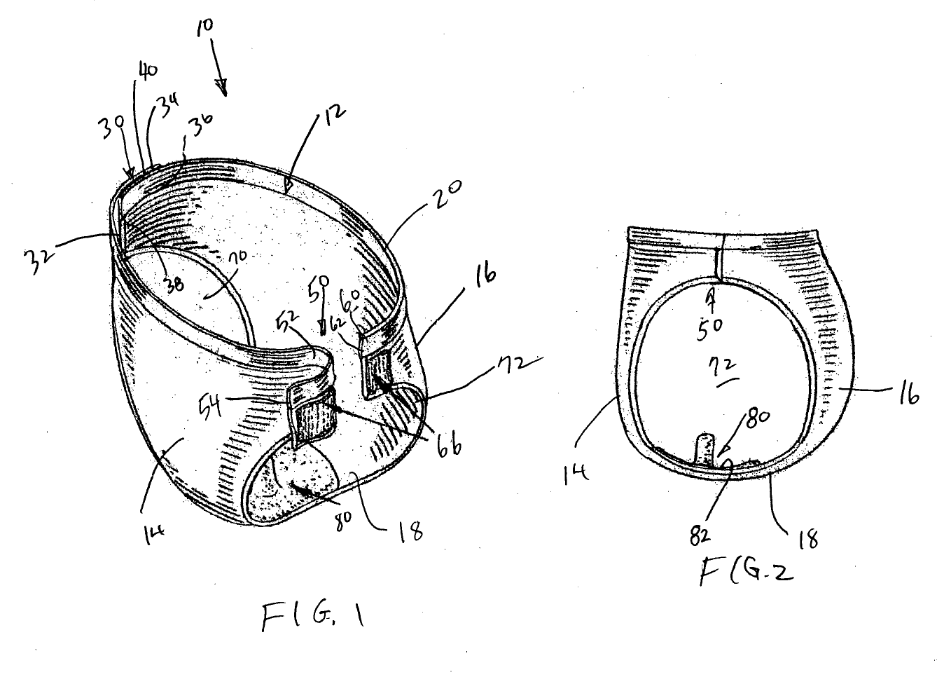

An elastic waistband 20 can encircle the body portion if suitable. A first flap portion 30 is located between the first portion and the second portion, and includes a first flap 32 which is unitary with the first portion and which has an end 34. First flap portion 30 further includes a second flap 36 unitary with the second portion and which has an end 38. First hook-and-loop fastening material 40 is located on the ends of the first and second flaps of the first flap portion. As shown in FIG. 1, the ends of the first and second flaps of the first flap portion are releasably connected together by the first hook-and-loop fastening material when the body section is worn.

The ends of flaps 32 and 36 can be moved with respect to each other to adjust the size of the panty for wearing. Furthermore, the ends of the flaps can be separated to quickly remove the panty without requiring the user to pull the panty down their legs. The panty can also be easily donned by separating the ends of flaps 32 and 36, placing the panty on the user and connecting the ends.

A second flap portion 50 is located between the first portion and the second portion and is spaced apart from the first flap portion. Second flap portion 50 includes a first flap 52 which is unitary with first portion 14 and has an end 54. Second flap portion 50 further includes a second flap 60 which is unitary with second portion 16 and has an end 62. Second hook-and-loop fastening material 66 is located on the ends of the first and second flaps of the second flap portion. Ends 54 and 62 of the first and second flaps of the second flap portion are releasably connected together by the second hook-and-loop fastening material when the body section is worn. Second flap portion 50 can be adjusted for size and opened and closed as discussed above for the first flap portion to permit easy donning and removing of the panty as well as easy size adjustment thereof.

First and second leg openings 70 and 72 are defined by first portion 14 and second portion 16 and crotch portion 18 and first and second flap portions 30 and 50 when the flaps of the flap portions are connected together. A feminine sanitary article, such as tampon 80, or the like is integral with the crotch portion. As can be seen in FIG. 2, the sanitary article extends above a plane 82 which contains the crotch section so the article is easily insertable when the panty is donned.

While various embodiments of the invention have been described, it will be apparent to those of ordinary skill in the art that many more embodiments and implementations are possible within the scope of this invention. Accordingly, the invention is not to be restricted except in light of the attached claims and their equivalents.

What is claimed is:

1. A disposable lined panty comprising:
A) a body section having
(1) a first portion that is a front portion when the body section is worn,
(2) a second portion that is a rear portion when the body section is worn,
(3) a crotch portion connecting the first portion to the second portion,
(4) a first flap portion located between the first portion and the second portion, the first flap portion including
(a) a first flap unitary with the first portion and having an end,
(b) a second flap unitary with the second portion and having an end,
(c) first hook-and-loop fastening material on the ends of the first and second flaps of the first flap portion, the ends of the first and second flaps of the first flap portion being releasably connected together by the first hook-and-loop fastening material when the body section is worn,
(5) a second flap portion located between the first portion and the second portion, the second flap portion being spaced apart from the first flap portion and including
(a) a first flap unitary with the first portion and having an end,
(b) a second flap unitary with the second portion and having an end,
(c) second hook-and-loop fastening material on the ends of the first and second flaps of the second flap portion, the ends of the first and second flaps of the second flap portion being releasably connected together by the second hook-and-loop fastening material when the body section is worn,
(6) first and second leg openings defined by the first portion and the second portion and the crotch portion and the first and second flap portions when the flaps of the flap portions are connected together; and
B) a feminine sanitary article integral with the crotch portion, the sanitary article extending above a plane containing the crotch section.

2. The disposable lined panty defined in claim 1 further comprising an elastic waistband encircling the body section.

3. A disposable lined panty comprising:
A) a body section having a crotch portion, and two flap portions, each flap portion including two flaps, each flap having hook-and-loop fastener material thereon, the flaps of each flap portion being releasably connected by the hook-and-loop fastener material when the body section is worn;
B) a feminine sanitary article integral with the crotch portion, the sanitary article extending above a plane containing the crotch section.
